(19) United States
(12) Patent Application Publication
(10) Pub. No.: US 2009/0275527 A1
(43) Pub. Date: Nov. 5, 2009

(54) SKIN AGING TREATMENT COMPRISING PAEONIFLORIN

(75) Inventors: Hyoung-Kook Park, Daejeon-city (KR); Jun-Man Lim, Daejeon-city (KR); Sang-Hwa Lee, Daejeon-city (KR); Sang-Jin Kang, Daejeon-city (KR); Wan-Goo Cho, Daejeon-city (KR)

Correspondence Address: SHERIDAN ROSS PC, 1560 BROADWAY, SUITE 1200, DENVER, CO, 80202, US

(73) Assignee: LG HOUSEHOLD & HEALTH CARE LTD., Seoul (KR)

(21) Appl. No.: 12/502,769

(22) Filed: Jul. 14, 2009

Related U.S. Application Data
(63) Continuation of application No. 11/571,906, filed on Jan. 10, 2007.

(30) Foreign Application Priority Data
May 10, 2004 (KR) 10-2004-0032641
Mar. 7, 2005 (KR) 10-2005-0018589
Apr. 28, 2005 (KR) 10-2005-0035482

(51) Int. Cl. A61K 31/7048; A61P 17/00

(52) U.S. Cl. 514/27

(57) ABSTRACT

The present invention relates to a skin aging treatment comprising paeoniflorin as an active ingredient. Since paeoniflorin is effective in significantly inhibiting and improving intrinsic skin aging, significantly inhibiting and improving DNA impairment and skin wrinkling caused by UV and improving existing wrinkles, a cosmetic composition and a pharmaceutical composition comprising paeoniflorin may become a very useful skin aging treatments.

### CROSS REFERENCE TO RELATED APPLICATION

This application is continuation of U.S. application Ser. No. 11/571,906 having a filing date of Jan. 10, 2007 which is a national stage application under 35 U.S.C. 371 of PCT Application No. PCT/KR2005/001310 having an international filing date of May 10, 2005, which designated the United States, which PCT application claimed the benefit of Korean Application Serial No. 10-2004-0032641, filed May 10, 2004; Korean Application Serial No. 10-2005-0018589, filed Mar. 7, 2005; and Korean Application Serial No.10-2005-0035482, filed Apr. 28, 2005, the entire disclosures of which are hereby incorporated herein by reference.

### BACKGROUND OF THE INVENTION

(a) Field of the Invention

The present invention relates to a skin aging treatment comprising paeoniflorin, which is effective in significantly inhibiting and improving intrinsic skin aging, significantly inhibiting and improving DNA impairment and skin wrinkling caused by UV and improving existing wrinkles, as an active ingredient.

(b) Description of the Related Art

Human skin is an important tissue playing a barrier role of protecting the human body from the external environment. Humans undergo skin aging throughout their lives. Skin aging can be classified into intrinsic aging and photoaging. Intrinsic aging is caused by the intrinsic, typically genetic, factors of the body. It is characterized by wrinkles and extended skin. Photoaging is caused by repeated exposure to UV along with intrinsic aging. Skin exposed to UV becomes rough, deeply and thickly wrinkled, with irregular hyperpigmentation, blood vessel expansion, hornification disorder, abnormal growth of the horny layer, modification of elastic fibers of the dermis and accumulation of degradation products. Also, UV accelerates decomposition of collagen and other elastic proteins in the dermis, aggravating skin damage and in severe cases, leads to skin cancer. In addition, UV is the cause of wrinkles, inelastic skin, dry skin, reduction of skin luster, rough skin, freckles, rupture of capillaries, skin discoloration, etc. (J. Pathol., 1997, 180; 80-89).

Skin aging is accompanied by the following changes. Connection of aged skin to the dermis becomes tight. The stratum granulosum and the stratum spinosum become thin. Contents of collagen and elastin reduce. Arrangement of cell layers becomes chaotic. Numbers of melanophores, Langerhans' cells and mast cells reduce. These phenomena can be confirmed by observing the cell histology (Optical microscope: OM, Electron microscope: EM, Aging skin, edited by J L Leveque & P G Agache, 1993, NY). In addition, aged skin experiences certain pattern changes in biochemistry, enzyme histology, etc. Particularly, skin metabolism enzymatic activity reduces, activity of oxidation-reduction enzyme systems weakens (SOD, GSH-Px) and oxidation-damaged products (MDA, free radicals, etc.) increase. These support such aging theories as error catastrophe theory and free radical theory.

Until now, research on skin aging has been mostly focused on photoaging and research on intrinsic aging has been insufficient. Anti-UVs that have been developed to prevent photoaging have many problems, including a lack of stability.

Paeoniflorin (C₂₃H₂₈O₁₁; M.W. 480.45) is one of the main components of the root of the peony family. It is a colorless crystalline substance and is also called peony saponin. It is known to be effective in expanding blood vessels, fighting against inflammation and hypersensitiveness and promoting immune activity.

Korean Patent Publication No. 2005-0017066 disclosed a skin care composition comprising resveratrol, which is derived from peony. Korean Patent Publication No. 2003-0004486 disclosed a cosmetic composition for preventing aging comprising a peony bark extract and a silk-tree extract. Korean Patent Publication No. 2002-0044266 disclosed a cosmetic composition for skin protection comprising a composite herb extract of white peony, etc. However, there has been no research on the effect of skin aging prevention and improvement thereof of paeoniflorin, as yet.

### SUMMARY OF THE INVENTION

It is an aspect of the present invention to provide a skin aging treatment comprising paeoniflorin, which is capable of safely and effectively improving skin aging, as an active ingredient.

It is another aspect of the invention to provide a cosmetic composition and/or a pharmaceutical composition comprising paeoniflorin as an active ingredient.

### DETAILED DESCRIPTION OF THE PREFERRED EMBODIMENTS

To attain the aspects, the present invention provides a skin aging treatment comprising paeoniflorin as an active ingredient.

The invention also provides a cosmetic composition for preventing skin aging comprising 0.001-10.0 wt % of paeoniflorin based on dry weight.

The invention further provides a pharmaceutical composition for preventing skin aging comprising 0.001-10.0 wt % of paeoniflorin based on dry weight.

Since paeoniflorin enhances activity and capability of skin cells, facilitates synthesis of collagen fibers thereby contributing to toughness and elasticity of skin tissue, reduces production of lipid peroxides thereby significantly preventing intrinsic skin aging, prevents DNA impairment and skin wrinkling caused by UV and effectively improves existing wrinkles, a cosmetic composition comprising paeoniflorin can be a very potent skin aging treatment.

Hereunder is given a detailed description of the present invention.

The present inventors completed this invention by finding out that paeoniflorin, which is safe for the human body, prevents and improves not only intrinsic skin aging but also skin photoaging caused by UV.

When applied on the skin of a hairless mouse, when prepared as an ointment or lotion for external application, paeoniflorin enhances activity and capability of skin cells, facilitates synthesis of collagen fibers thereby contributing to toughness and elasticity of skin tissue, reduces production of lipid peroxides thereby significantly preventing intrinsic skin aging, significantly prevents DNA impairment and skin wrinkling caused by UV and effectively improves existing wrinkles. It is also safe and has no side effects.

Paeoniflorin may be purchased or purified and separated by any known method. In a preferred embodiment of the present invention, paeoniflorin was directly isolated from the root of Chinese peony. The purification process of paeoniflorin comprises the steps of: (a) adding 1-10 volume equivalents of ethanol or a 50-95% aqueous ethanol solution to the root of Chinese peony (Paeonia lactiflora Pallas) for extraction; (b) concentrating the extract, removing remaining ethanol and adding water of that amount; (c) adding ethyl ether of the same volume to the resultant suspension for phase separation and separating the water layer; (d) concentrating the water layer, pouring it to a silica gel column and performing chromatography with a mixture of chloroform and acetone; (e) pouring the eluent to an octadecylsilylated silica gel column and performing chromatography with a mixture of trifluoroacetic acid and methanol; and (f) separating the eluent and performing distillation under reduced pressure to obtain paeoniflorin. This process can be easily performed by one skilled in the art. Modification or substitution thereto in part belongs within the scope of the present invention.

The skin aging treatment of the present invention may comprise paeoniflorin alone. However, depending on preparation form and method of application, it may further comprise a pharmaceutically available excipient. In that case, paeoniflorin is comprised at 0.001-10.0 wt %, preferably at 0.01-1.0 wt %, based on dry weight. If the content of the active ingredient is below 0.001 wt %, an obvious skin aging protection or improvement effect cannot be expected. Otherwise, if it exceeds 10.0 wt %, the effect may not increase in spite of the increased content, so that it may be uneconomical. However, it is desirable to adjust the content of the active ingredient depending on method of application and purpose of using the skin aging treatment. The pharmaceutically available excipient may be glycerine, starch, lactose, water, an alcohol, propylene glycol, salicylic acid, dihydroacetic acid or and physiological saline, but is not limited to these.

The skin aging treatment of the present invention may be administered orally or non-orally. Preferably, it is administered non-orally, for example transdermally. It is prepared into an adequate preparation form depending on how it is to be administered. For example, it may be prepared into plasters, granules, lotions, powders, syrups, liquids or solutions, aerosols, ointments, fluid extracts, emulsions, suspensions, infusions, tablets, injections, capsules, pills, etc., but is not limited to these.

Preferably, the administration dose of the skin aging treatment of the present invention is determined considering purpose of use, part of the body to which it is to be administered, method of administration, age, sex and physical conditions of the patient, absorptivity of the active ingredient in the body, ratio of inactivity, compatible drugs, and so on. For example, a dose of 0.01 mg/kg (body weight) to 500 mg/kg (body weight), based on the active ingredient, may be administered each day.

The skin aging treatment of the present invention may also be used as a cosmetic composition or pharmaceutical composition. Thus, the present invention provides a cosmetic composition or pharmaceutical composition for preventing skin aging comprising paeoniflorin as an active ingredient.

The cosmetic composition or pharmaceutical composition may comprise 0.001-10.0 wt %, preferably 0.01-1.0 wt %, of paeoniflorin, based on dry weight. However, it is desirable to adjust the content depending on preparation form or contents of other ingredients. If the content of paeoniflorin in the cosmetic composition or pharmaceutical composition is below 0.001%, it is difficult to expect practical prevention or improvement of skin aging. Otherwise, if it exceeds 10.0 wt %, the amount of paeoniflorin is excessive for its effect, so that it is uneconomical. The cosmetic composition or pharmaceutical composition may comprise a conventional cosmetic ingredient or any pharmaceutically available ingredient, such as an excipient, a diluent, etc., in addition to paeoniflorin. For example, it may comprise diethyl sebacate, spermaceti, vaseline, polyoxyethyleneoleyl ether phosphate, sodium benzoate, stearic acid, cetanol, sorbitan monostearate, mineral oil, triocatnoate, triethanolamine, carbomer, glycerine, propylene glycol, purified water, ethanol, polyoxyethylene-hardened caster oil, methyl p-oxybenzoate, 1,3-butylene glycol, sodium hyaluronate, etc.

The cosmetic composition of the present invention may be used for basic cosmetics, makeup cosmetics, body cosmetics, hair cosmetics, scalp cosmetics, shaving cosmetics or oral cosmetics. Examples of the basic cosmetics are cream, essence, pack, massage cream, emulsion, etc. Examples of the makeup cosmetics are foundation, makeup base, lipstick, eye shadow, eye liner, mascara, eyebrow pencil, etc. Examples of the body cosmetics are soap, liquid cleaner, bath treatment, sunscreen cream, sun oil, etc. Examples of the hair cosmetics are shampoo, rinse, hair treatment, hair mousse, hair liquid, pomade, hair dye, hair bleacher, color rinse, etc. Examples of the scalp cosmetics are hair tonic, scalp treatment, etc. Examples of the shaving cosmetics are aftershave lotion, shaving cream, etc. Examples of the oral cosmetics are toothpaste, mouth wash, etc.

Hereinafter, the present invention is described in detail with reference to examples. However, the following examples are only for the understanding of the present invention and they do not limit the present invention.

### EXAMPLES

### Preparation Example 1

### Separation and Purification of Paeoniflorin

The root of Chinese peony (Paeonia lactiflora Pallas) was pulverized 5 volume equivalents of an extraction solvent comprising 75% ethanol and water was added to the pulverized root. Extraction was performed 3 times, each for 3 hours, and then the extract was concentrated. Remaining ethanol was removed from the concentrate and water of the same volume was added. The mixture was heated to dissolve it. Then, ethyl ether of the same volume was added to partition the solution three times. The water layer was concentrated. The concentrate was put in a 200-300 mesh silica gel column. A mobile phase comprising chloroform and acetone (4:1) was used to separate the concentrate. The eluent was concentrated and dried. The resultant dry product contained about 70% of paeoniflorin. It was suspended in water and put in a 10 mm (diameter)×250 mm (length), 4 μm column filled with octadecylsilylated silica gel. A mixture solvent comprising 0.5% TFA (trifluoroacetic acid) and methanol (volume ratio=75:25) was used as the mobile phase. Detection of paeoniflorin was performed with a UV detector (230 nm). The mobile phase was fed at a rate of 2.5 mL/min. The main peak detected over 18 and 20 minutes was separated repeatedly. The obtained partition was distilled under reduced pressure to obtain paeoniflorin.

The resultant paeoniflorin was analyzed with an LCQ mass spectrometer (Finnigan, U.S.) and an NMR spectrometer (Bruker).

Table 1 below shows the mass analysis result and Table 2 below shows the NMR analysis result. The structure of paeoniflorin was identified from these results (Formula 1, C₂₃H₂₈O₁₁).

TABLE 1

| | | Estimated | |
|---|---|---|---|
| Sample | [M + Na]+ | molecular weight | Remarks |
| Paeoniflorin | 503 | 480 | 983 = [2M + Na]+ |

TABLE 2

| | Classification | ¹H | ¹³C |
|---|---|---|---|
| 1 | Q | — | 89.3 |
| 2 | CH₂ | 2.48, 1.95 | 23.3 |
| 3 | CH | 2.59 | 43.9 |
| 4 | Q | — | 106.3 |
| 5 | CH₂ | 2.19, 1.82 | 44.4 |
| 6 | Q | — | 87.2 |
| 7 | Q | — | 71.6 |
| 8 | CH₃ | 1.36 | 19.6 |
| 10 | CH₂ | 1.64, 1.53 | 102.2 |
| 12 | CH₂ | 1.51, 1.40 | 62.8 |
| 1′ | Q | — | 131.1 |
| 2′ | CH * 2 | 8.04 | 130.6 |
| 3′ | CH * 2 | 7.47 | 129.6 |
| 4′ | CH | 7.60 | 134.4 |
| 1″ | CH | 4.53 | 100.1 |
| 2″ | CH | 3.37-3.18 | 77.8 |
| 3″ | CH | 3.37-3.18 | 77.9 |
| 4″ | CH | 3.37-3.18 | 72.1 |
| 5″ | CH | 3.37-3.18 | 74.9 |
| 6″ | CH | 3.85, 3.61 | 61.7 |

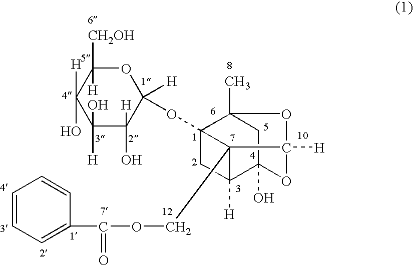

HPLC analysis of the resultant paeoniflorin showed that it had a purity of at least 99%.

### Testing Example 1

### Effect on Fibroblast

Paeoniflorin's effect on cell activity was tested.

Keratinocytes and fibroblasts are mainly located in the dermis and the epidermis and play parts in key functions of the epidermis and the dermis. Thus, keratinocytes and fibroblasts are used as test cells in research of cell physiotoxicology and anti-aging.

Skin tissue was taken from a red skin mouse 3 days or less old. Hypodermic tissue was removed and the skin was treated with 0.25% trypsin for 12 hours. The skin was separated into the epidermis and the dermis. The epidermis was cultured to 75% confluence in an epidermis culture medium (K-SFM). The dermis was further treated with trypsin at 37° C. for 2 hours. Fibroblasts were taken from the dermis and cultured to 75% confluence in a DMEM culture medium containing 10% serum.

Paeoniflorin prepared in Preparation Example 1 was dissolved in the cultured keratinocytes and fibroblasts and treated to 0.0004 M. Cell count was measured by MTT [3-(4,5-dimethylthiazol-2-yl)-2,5-diphenyltetrazolium bromide)] analysis to determine cell activity. A non-treated group and a group treated with 0.002 M vitamin C were tested as control groups. Effect of paeoniflorin on the fibroblasts is given in Table 3 below.

TABLE 3

| | Increase in cell activity (%) |
|---|---|
| Non-treated | 0 |
| 0.0004 M paeoniflorin | 25.7 |
| 0.002 M vitamin C | 14.4 |

As seen in Table 3, paeoniflorin significantly facilitates cell proliferation of fibroblasts.

### Testing Example 2

### Prevention Against Skin Cell Death and Aging

Cell growth and division status was confirmed by specifically staining cell DNA.

β-Gal is a material for specifically marking aged skin cells. Young cells and cells in the resting phase are left intact and only aged cells are specifically stained. Cultured keratinocytes and fibroblasts were treated with a minimum amount of X-Gal, respectively, and stained at 37 for 24 hours. Aged skin cells were stained to deep blue in 2-4 hours and gave the best result in 12-16 hours (see Proc. Natl. Acad. Sci. USA, 1995, 92: 9363-7). Then, stained cells (aged cells), living cells and dead cells were counted with a flow cytometer. The results are given in Table 4 below. In Table 4, “blank” refers to the control group containing no cells.

TABLE 4

| | | Cell growth | Cell death | Cell aging |
|---|---|---|---|---|
| Cells | Treatment (M) | rate | rate | rate |
| Keratinocytes | Blank | — | — | — |
| | Paeoniflorin | 55.3% | 12.3% | 24.7% |
| | 0.0005 M | | | |
| | Vitamin E | 23.3% | 10.5% | 20.1% |
| | 0.0004 M | | | |
| Fibroblasts | Blank | — | — | — |
| | Paeoniflorin | 18.4% | 11.5% | 17.2% |
| | 0.0005 M | | | |
| | Vitamin E | 9.96% | 15.4% | 22.8% |
| | 0.0004 M | | | |

As seen in Table 4, paeoniflorin was confirmed to reduce cell death rate and cell aging rate of keratinocytes and fibroblasts. Especially, paeoniflorin was confirmed to improve cell growth rate better than vitamin E. Thus, it was identified that paeoniflorin is superior to vitamin E in preventing aging of skin cells.

### Testing Example 3

### Changes in Contents of Lipid Peroxides and Collagen

Production of lipid peroxides and change in collagen content were measured for animal skin in order to confirm the skin aging effect of paeoniflorin.

1) Sample preparation

10 g of stearic acid (C₁₈H₃₆O₂), 70 mL of water and 20 mL of glycerol, each heated to 80, were mixed homogeneously. After stirring for about 1 minute, 3 (w/v)% of paeoniflorin or vitamin E (positive control group) was added. After adding 0.1 (w/v)% of methyl paraben, stirring was performed for 1 minute. 1 mL of KOH was slowly added. After considerable emulsification had proceeded, stirring was performed until the temperature reached about 50.

2) Testing

Guinea pigs were grouped, each group consisting of 3 males and 3 females. An area of 3×3 cm² was shaved from the back of each guinea pig. For each guinea pig, the left side was treated with the sample prepared above and the right side was treated as a negative control group and a positive control group. Application was performed at 8:00 H and 16:00 H each day. Application was performed for 30 days, at about 5 mg/cm² each. On the 31st day, skin was taken from the back of each animal. Subcutaneous fat tissue was removed and collagen and lipid peroxide contents were measured with an electron microscope and an optical microscope. 3) Result

a) Observation with the naked eyes

The groups treated with the sample showed significant apparent improvement, such as soft and shiny skin. The effect was outstanding compared with the positive control group treated with vitamin E. On the contrary, the blank control group and the negative control group not treated with paeoniflorin showed no apparent improvement.

b) Histological observation—collagen content

Back skin tissue was impregnated with paraffin and stained with HE (hematoxylin-eosin). Change of the skin tissue was observed. Skin collagen content was determined by adding 6 N hydrochloric acid to skin and measuring the HYP (hydroxyproline) content according to the chloramine-T oxidation method.

The group to which paeoniflorin was applied showed no significant change in the epidermis, but an increase in collagenous fiber bundles was observed in the dermis. Table 5 below shows the effect of paeoniflorin on the collagenous fiber bundles in the dermis. It can be seen that paeoniflorin increases the collagen content in the dermis, thereby improving skin elasticity.

TABLE 5

| | Increase of collagen content | Increase of collagenous fiber |
|---|---|---|
| Sample | in dermis (%) | bundles (%) |
| Blank | — | — |
| Paeoniflorin | 35.92* | 18.1* |
| Vitamin E | 29.86* | 10.4* |

*Compared with the positive control group, P < 0.05

Expansion of RER (rough endoplasmie reticulum) and increase of mitochondria were also observed in the dermis, which confirms that paeoniflorin enhances activity and capability of skin cells.

c) Histological observation—lipid peroxide content

SOD (superoxide dismutase) and GSH-Px (total glutathione peroxidase) are important antioxidative enzymes in living organisms. They play important roles in gaseous oxidation and antioxidation balance. Since they offer antioxidative effects by removing oxygen free radicals, activity of SOD and GSH-Px can be interpreted by the capability of removing oxygen free radicals in the body. MDA (malondialdehyde) is a kind of lipid peroxide.

MDA was detected using thiobarboturic acid. GSH-Px and SOD contents were measured by DNTB (5,5′-dithio-bis-2-nitrobenzoic acid) and nitrate reduction methods, respectively. The result is given in Table 6 below.

Content=(Absorptivity of treated group−Absorptivity of blank group)/(Absorptivity of blank group)×100 [Equation 1]

TABLE 6

| Sample | MDA | SOD | GSH-Px |
|---|---|---|---|
| Blank | — | — | — |
| Paeoniflorin | 29.57* | 8.58* | 29.51* |
| Vitamin E | 26.71* | 7.17* | 28.60* |

As seen in Table 6, paeoniflorin improves SOD and GSH-Px activity in the skin tissue and inhibits production of lipid peroxides.

### Testing Example 4

### Prevention of DNA Impairment Caused by UV

Prevention effect of DNA impairment caused by UV of paeoniflorin prepared in Preparation Example 1 was determined for normal human skin keratinocytes.

Cultured normal human keratinocytes were treated with paeoniflorin of concentrations thereof for 12 hours. The culture medium was removed and the cells were washed with PBS. 60 mJ UVB was irradiated and DNA impairment of the cells was checked. If there is DNA strand breakage inside the cell nucleus, an extended tail is shown toward the positive electrode during electrophoresis. Thus, impairment of DNA can be easily detected by electrophoresis. The average of tail moment of 50 randomly selected cells was used as a control value. The tail moment of paeoniflorin-treated cells was averaged. The control value and the resultant test value were put in Equation 2 below to calculate paeoniflorin's prevention effect of DNA impairment caused by UV.

Prevention effect (%)=(Control value−Test value)/(Control value)×100 [Equation 2]

TABLE 7

| Concentration of | Average tail moment | |
|---|---|---|
| paeoniflorin | (n = 50) | Prevention effect (%) |
| Negative control group | 0.790 ± 0.662 | — |
| UV-irradiated positive | 9.566 ± 2.828 | — |
| control group | | |
| 0.0001% | 7.951 ± 1.887 | 16.9 |
| 0.001% | 7.710 ± 2.161 | 19.4 |
| 0.01% | 7.409 ± 2.395 | 22.6 |
| 0.1% | 7.320 ± 2.534 | 23.5 |

Table 7 shows paeoniflorin's prevention effect of DNA impairment caused by UV. Paeoniflorin shows at least 16.9% DNA impairment prevention effect in the treatment concentration range of 0.0001% to 0.1%.

### Testing Example 5

### Prevention of DNA Impairment Caused by UV (In Vivo)

Paeoniflorin's prevention of DNA impairment caused by UV was confirmed by animal tests.

Paeoniflorin dissolved in 80% ethanol was applied to the back of hairless mice. After 6 days, 1 J UVB was irradiated. Skin cells were taken and DNA impairment was measured. The results are given in Table 8 below.

TABLE 8

| Concentration of | Average tail moment | |
|---|---|---|
| paeoniflorin | (n = 50) | Prevention effect (%) |
| Negative control group | 2.752 ± 2.720 | — |
| UV-irradiated positive | 25.348 ± 6.049 | — |
| control group | | |
| 1% | 18.363 ± 5.463 | 27.5 |
| 0.1% | 18.930 ± 5.969 | 25.3 |
| 0.01% | 14.968 ± 5.883 | 40.9 |

As seen in Table 8, the DNA impairment prevention effect increased as the concentration of paeoniflorin increased. However, in animal tests, the result may not be proportional to the concentration over a specific concentration. That is, although the prevention effect was proportional up to 0.01%, it may not be so over 0.01%.

Paeoniflorin was confirmed to effectively prevent DNA impairment caused by UV in the animal test. This implies that paeoniflorin can show a strongly DNA impairment prevention effect in living animals, as well as in cultured cells.

### Testing Example 6

### Prevention of Wrinkles Caused by UV

Prevention effect of wrinkles caused by UV of paeoniflorin prepared in Preparation Example 1 was evaluated as follows using hairless mice. UV was irradiated to hairless mice with a solar stimulator at 2MED (minimum erythema dose). Irradiation was performed for 12 weeks, three days a week. Of the total of 10 mice, 0.2% paeoniflorin dissolved in 1,3-butylene glycol was applied to the sample treatment group and only 1,3-butylene glycol was applied to the control group. Wrinkles of each group were compared 12 weeks later. Evaluation was performed with three ratings: not improved compared to the control group; slightly improved; and significantly improved. The results are shown in Table 9 below.

TABLE 9

| | | | Significantly |
|---|---|---|---|
| Sample | Not improved | Slightly improved | improved |
| Control group | 9 | 1 | 0 |
| Paeoniflorin (0.2%) | 0 | 4 | 6 |

It was confirmed that paeoniflorin effectively prevents wrinkling caused by UV in the skin of living hairless mice.

### Example 1 and Comparative Example 1

### Preparation of Skin Ointment

Skin ointment containing paeoniflorin was prepared as in Table 10.

TABLE 10

Contents (wt %)

| | | Comparative |
|---|---|---|
| Composition | Example 1 | Example 1 |
| Paeoniflorin | 1.0 | — |
| Diethyl sebacate | 8 | 8 |
| Spermaceti | 5 | 5 |
| Polyoxyethyleneoleyl ether phosphate | 6 | 6 |
| Sodium benzoate | Adequate | Adequate |
| Vaseline | to 100 | to 100 |

### Example 2 and Comparative Example 2

### Preparation of Cream

Cosmetic cream containing paeoniflorin was prepared as in Table 11.

TABLE 11

Contents (wt %)

| | | Comparative |
|---|---|---|
| Composition | Example 2 | Example 2 |
| Paeoniflorin | 0.5 | — |
| Stearic acid | 1.0 | 1.0 |
| Cetanol | 2.0 | 2.0 |
| PEG-20 | 1.0 | 1.0 |
| Sorbitan monostearate | 1.0 | 1.0 |
| Mineral oil | 10.0 | 10.0 |
| Triocatnoate | 5.0 | 5.0 |
| Triethanolamine | 0.5 | 0.5 |
| Carbomer | 0.2 | 0.2 |
| Glycerine | 5.0 | 5.0 |
| Propylene glycol | 3.0 | 3.0 |
| Antiseptic | Adequate | Adequate |
| Flavor | Adequate | Adequate |
| Purified water | to 100 | to 100 |

### Example 3 and Comparative Example 3

Preparation of Soft essence

Soft essence containing paeoniflorin was prepared as in Table 12.

TABLE 12

Contents (wt %)

| | | Comparative |
|---|---|---|
| Composition | Example 3 | Example 3 |
| Paeoniflorin | 0.2 | — |
| Ethanol | 10.0 | 10.0 |
| Polyoxyethylene-hardened caster oil | 1.0 | 1.0 |
| Methyl p-oxybenzoate | 0.2 | 0.2 |
| Glycerine | 5.0 | 5.0 |
| 1,3-Butylene glycol | 6.0 | 6.0 |
| Flavor | Adequate | Adequate |
| Pigment | Adequate | Adequate |
| Purified water | to 100 | to 100 |

### Example 4 and Comparative Example 4

### Preparation of Essence

Essence containing paeoniflorin was prepared as in Table 13.

TABLE 13

Contents (wt %)

| | | Comparative |
|---|---|---|
| Composition | Example 4 | Example 4 |
| Paeoniflorin | 1 | — |
| Propylene glycol | 10.0 | 10.0 |
| Glycerine | 10.0 | 10.0 |
| Sodium hyaluronate solution (1%) | 5.0 | 15.0 |
| Ethanol | 5.0 | 5.0 |
| Polyoxyethylene-hardened caster oil | 1.0 | 1.0 |
| Methyl p-oxybenzoate | 0.1 | 0.1 |
| Carbomer | 0.3 | 0.3 |
| Triethanolamine | 0.4 | 0.4 |
| Flavor | Adequate | Adequate |
| Purified water | to 100 | to 100 |

### Example 5 and Comparative Example 5

### Preparation of Pack

Pack containing paeoniflorin was prepared as in Table 14.

TABLE 14

Contents (wt %)

| | | Comparative |
|---|---|---|
| Composition | Example 5 | Example 5 |
| Paeoniflorin | 0.3 | — |
| Glycerine | 5.0 | 5.0 |
| Propylene glycol | 4.0 | 4.0 |
| Polyvinylalcohol | 15.0 | 15.0 |
| Ethanol | 8.0 | 8.0 |
| Polyoxyethylene-hardened caster oil | 1.0 | 1.0 |
| Polyoxyethylene ethyl oleate | 1.0 | 1.0 |
| Methyl p-oxybenzoate | 0.2 | 0.2 |
| Flavor | Adequate | Adequate |
| Pigment | Adequate | Adequate |
| Purified water | to 100 | to 100 |

### Example 6 and Comparative Example 6

### Preparation of Nutritional Essence

Nutritional essence containing paeoniflorin was prepared as in Table 15.

TABLE 15

Contents (wt %)

| | | Comparative |
|---|---|---|
| Composition | Example 6 | Example 6 |
| Paeoniflorin | 0.5 | — |
| Polyoxyethylene-hardened caster oil | 1.0 | 1.0 |
| Methyl p-oxybenzoate | Adequate | Adequate |
| Glycerine | 6.0 | 6.0 |
| 1,3-Butylene glycol | 5.0 | 5.0 |
| Carbomer | 0.2 | 0.2 |
| Triethanolamine | 0.3 | 0.3 |
| Propylene glycol | 5.0 | 5.0 |
| Ethanol | 3.2 | 3.2 |
| Carboxyvinyl polymer | 0.1 | 0.1 |
| Pigment | Adequate | Adequate |
| Flavor | Adequate | Adequate |
| Purified water | to 100 | to 100 |

### Testing Example 7

### Wrinkling Improvement Effect

The wrinkling improvement effect of the ointments of Example 1 and Comparative Example 1 and the creams of Example 2 and Comparative Example 2 was tested on healthy women 35 to 50 years old.

40 women 35 to 50 years old were divided into two groups, with 20 in each. For the first group, the ointments of Example 1 and Comparative Example 1 were applied on half of the face of each. For the second group, the creams of Example 2 and Comparative Example 2 were applied on half of the face of each. Application on left or right side of the face was randomly determined. Both researchers and subjects did not know which ointment or cream was applied on which side of the of face. The subjects applied the test cosmetics for 8 weeks, two times a day, after washing the face.

8 weeks later, wrinkling improvement was evaluated with the naked eyes and image analysis. Wrinkling improving and improvement in elasticity were observed with the naked eye and evaluated as not improved, slightly improved or significantly improved. The results are given in Table 16.

TABLE 16

| | | Slightly | Significantly | |
|---|---|---|---|---|
| Sample | Not improved | improved | improved | P value |
| Comparative | 13 | 5 | 2 | <0.05 |
| Example 1 | | | | |
| Example 1 | 4 | 8 | 8 | |
| Comparative | 11 | 6 | 3 | <0.05 |
| Example 2 | | | | |
| Example 2 | 3 | 8 | 9 | |

Image analysis was performed by comparing images of replicas taken under the eye before and after testing (Xantopren, Bayer). Density of wrinkles was measured by 2-dimensional image analysis. Averaged decrease in wrinkle density is given in Table 17.

TABLE 17

| Sample | Decrease in wrinkle density (%) | P value |
|---|---|---|
| Comparative | 8% | <0.05 |
| Example 1 | | |
| Example 1 | 43% | |
| Comparative | 7% | <0.05 |
| Example 2 | | |
| Example 2 | 45% | |

As seen in Tables 16 and 17, the ointment and the cosmetic cream containing paeoniflorin showed a wrinkling improvement effect in at least 16 subjects out of 20 in the observation with the naked eye. They showed a superior wrinkle reduction effect in image analysis with no side effects. Thus, it can be seen that paeoniflorin is outstandingly effective in wrinkling improvement while being safe.

As apparent from the above description, paeoniflorin enhances activity and capability of skin cells and facilitates synthesis of collagen fibers, thereby contributing to toughness and elasticity improvement of the skin tissue. In addition, it reduces production of lipid peroxides, thereby significantly preventing intrinsic skin aging, significantly prevents DNA impairment and skin wrinkling caused by UV and is effective in improving existing wrinkles. Therefore, a cosmetic composition comprising paeoniflorin can become a very useful skin aging treatment.

What is claimed is:

1. A method for treating skin aging comprising applying a composition to a skin, wherein the composition comprises paeoniflorin as an active ingredient.

2. The method of claim 1, wherein the paeoniflorin prevents DNA impairment and skin wrinkling caused by UV or improving existing wrinkles.

3. The method of claim 1, wherein the composition comprises 0.001-10.0 wt % of paeoniflorin, based on dry weight.

4. The method of claim 1, wherein the composition is a cosmetic composition.

5. The method of claim 4, wherein the cosmetic composition is selected from the group consisting of cream, essence, pack, massage cream, emulsion, foundation, makeup base, lipstick and eye shadow.

6. The method of claim 1, wherein the composition is a pharmaceutical composition.
